(19) United States
(12) Patent Application Publication
(10) Pub. No.: US 2024/0216701 A1
(43) Pub. Date: Jul. 4, 2024

(54) HEADER SELF HEALING SELF PLUG & RELATED FEATURES

(71) Applicant: Cardiac Pacemakers, Inc., St. Paul, MN (US)

(72) Inventors: James Michael English, Cahir (IE); Moira B. Sweeney, St. Paul, MN (US)

(21) Appl. No.: 18/398,423

(22) Filed: Dec. 28, 2023

Related U.S. Application Data
(60) Provisional application No. 63/435,878, filed on Dec. 29, 2022.

(51) Int. Cl. A61N 1/375

(52) U.S. Cl. CPC A61N 1/3752; A61N 1/37512

(57) ABSTRACT

An implantable medical device can include a housing including electronic devices within the housing; a header attached to the housing and including one or more bores configured to receive a lead; and a set screw positioned in the header and configured to clamp to a lead inserted to the bore; wherein, a gel is inserted into a cavity around a head of the set screw.

### CLAIM OF PRIORITY

This application claims the benefit of U.S. Provisional Application No. 63/435,878 filed on Dec. 29, 2022, which is hereby incorporated by reference in its entirety.

### TECHNICAL FIELD

Various embodiments described herein relate to apparatus, systems, and methods associated with implantable medical devices.

### BACKGROUND

An ambulatory medical device, such as an implantable medical device (IMD), can be configured for implant in a subject, such as a patient. An IMD can be configured to be coupled to a patient's heart such as via one or more implantable leads. Such an IMD can obtain diagnostic information or generate therapy to be provided to the patient, such as via the coupled implantable lead.

In one configuration, IMDs have a header that is coupled to a container that houses much of the electronics of the IMD. The header can be used to couple a conductor of the lead with circuitry within the implantable device. The lead can be attached to the header using a set screw configuration. It is desirable that there is no fluid or contaminant flow into the set screw cavity when tightening the set screw for securing the lead in the header.

### SUMMARY

Example 1 can include subject matter such as an implantable device. An implantable medical device can include a housing including electronic devices within the housing; a header attached to the housing and including one or more bores configured to receive a lead; and a set screw positioned in the header and configured to clamp to a lead inserted to the bore; wherein, a gel is inserted into a cavity around a head of the set screw.

In Example 2, the subject matter of Example 1 can optionally include a semi-closed rubber grommet positioned in the cavity and positioned above the gel.

In Example 3, the subject matter of any one or more of Examples 1-2 can optionally include the gel being configured to fill the cavity.

In Example 4, the subject matter of any one or more of Examples 1-3 can optionally include the gel being a low viscosity gel.

In Example 5, the subject matter of any one or more of Examples 1-4 can optionally include the gel having a viscosity of less than about 1,000 cP (1,000 mPa*s).

In Example 6, the subject matter of any one or more of Examples 1-5 can optionally include the gel being a medical grade, silicone gel.

In Example 7, the subject matter of any one or more of Examples 1-6 can optionally include the set screw being configured to be tightened and loosed using a wrench.

In Example 8, the subject matter of any one or more of Examples 1-7 can optionally include the gel being reflowable so as to backfill the cavity after the wrench removed from the cavity.

In Example 9, the subject matter of any one or more of Examples 1-8 can optionally include the set screw being positioned in the cavity in the header so as to be oriented transversely to a longitudinal axis of the bore.

In Example 10, the subject matter of any one or more of Examples 1-9 can optionally include a wrench guiding cap positioned in the cavity over the gel.

In Example 11, the subject matter of any one or more of Examples 1-10 can optionally include the wrench guiding cap including a funnel shaped upper surface leading to a through hole in the wrench guiding cap.

Example 12 can include subject matter including a method for fastening a set screw in a header, the method can include inserting a wrench into a cavity in a header such that the wrench passes through a rubber grommet and then passes through a gel into a head of the set screw; tightening the set screw using the wrench; removing the wrench from the cavity; and the gel backfilling and sealing the cavity.

In Example 13, the subject matter of any one or more of Examples 1-12 can optionally include the gel being a low viscosity gel.

In Example 14, the subject matter of any one or more of Examples 1-13 can optionally include the gel having a viscosity of less than about 1,000 cP (1,000 mPa*s).

In Example 15, the subject matter of any one or more of Examples 1-14 can optionally include the gel being a medical grade, silicone gel.

In Example 16, the subject matter of any one or more of Examples 1-15 can optionally include a wrench guiding cap positioned in the cavity over the gel.

In Example 17, the subject matter of any one or more of Examples 1-16 can optionally include the wrench guiding cap including a funnel shaped upper surface leading to a through hole in the wrench guiding cap.

Example 18 can include subject matter including a method of manufacturing a header for an implantable device, the method can include placing a set screw in a header; inserting a gel into a cavity over and around a head of the set screw; and inserting a rubber grommet into the cavity over the gel.

In Example 19, the subject matter of any one or more of Examples 1-18 can optionally include inserting the gel comes after inserting the rubber grommet.

In Example 20, the subject matter of any one or more of Examples 1-19 can optionally include the gel being a low viscosity medical grade silicone gel.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

FIG. 1 shows an example implantable medical device, in accordance with one embodiment.

FIG. 2 shows a cross-section of a set screw within the header, in accordance with one embodiment.

FIG. 3 shows a side view of FIG. 2 with a wrench inserted into the into the set screw, in accordance with one embodiment.

FIG. 4 shows a cross-section of a set screw within the header, in accordance with one embodiment.

FIG. 5 shows a method for fastening a set screw in a header, in accordance with one embodiment.

FIG. 6 shows a method of manufacturing a header for an implantable device, in accordance with one embodiment.

### DETAILED DESCRIPTION

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

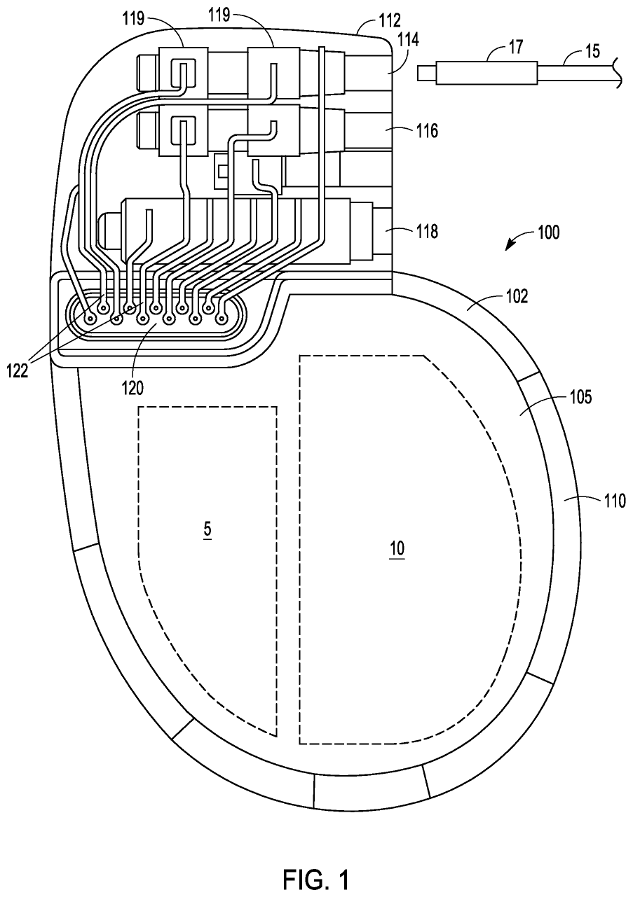

FIG. 1 shows an implantable system 100 including an implantable medical device 102, in accordance with one embodiment. The implantable medical device 102 includes a pulse generator 105 and at least one implantable lead 15. The pulse generator 105 includes a housing 110 and a header 112 mounted to the housing 110. The pulse generator 105 can be implanted into a subcutaneous pocket made in the wall of a patient's chest. Alternatively, the pulse generator 105 can be placed in a subcutaneous pocket made in the abdomen, or in other locations. Pulse generator 105 can include electronic devices such as a power supply 5 including a battery, a capacitor, and other components housed in the housing 110. The pulse generator 105 can further include other electronic devices such as microprocessors 10 to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

The header 112 can include one or more bores 114, 116, 118 to receive an implantable lead 15. The implantable lead 15 can include electrodes on a distal end to provide therapy to a body and include a terminal pin 17 on the proximal end to couple to the bore 114, 116, 118. At least one electrical conductor is disposed within the lead 15 and extends from the proximal end to the electrode. The electrical conductor carries electrical currents and signals between the pulse generator 105 and the electrode.

Contacts on the terminal pin 17 can electrically contact electrical contacts 119 within the bores 114, 116, 118 to allow signals and therapy to be delivered to and from electrodes in a body to the electronics 5, 10 within the housing 110. The contacts 119 can be connected by wires 122 to a feedthrough assembly 120 to electrically communicate between the lead 15 and the electronics within the housing 110.

In one example, the header 112 can be formed from a polymer material. A polymer can provide a number of desirable features, such as biocompatibility, strength, resilience, and ease of manufacturing. In one example, the header 112 is molded separately from the housing 110, and later bonded to the housing 110 using an adhesive. In a second example, the header 112 can be molded in place (overmolded) and contacts a surface of the housing 110 during a curing or hardening process.

In other embodiments, the implantable system 100 can also be suitable for use with implantable electrical stimulators, such as, but not limited to, neuro-stimulators, skeletal stimulators, central nervous system stimulators, or stimulators for the treatment of pain.

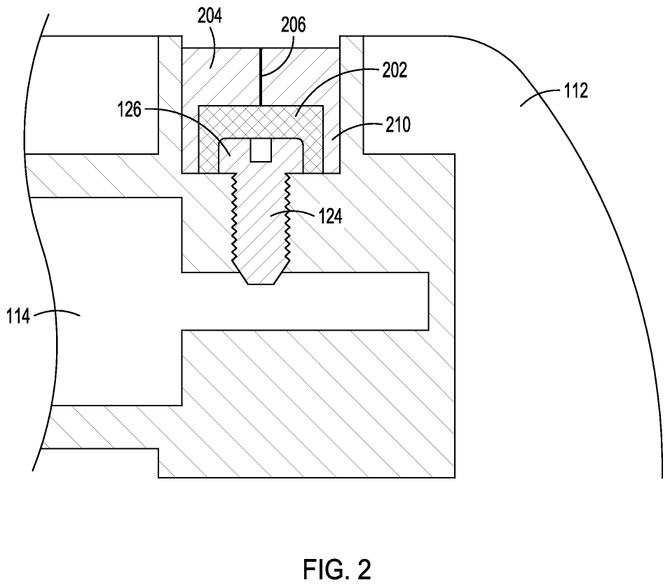

FIG. 2 shows a cross section of a set screw 124 within the header 112, in accordance with one embodiment. As noted, the header 112 can further include the set screw 124 which is configured to clamp to the lead terminal pin 17 (FIG. 1) when the terminal pin 17 is inserted to one of the bores 114, 116, 118. The set screw 124 is positioned in a cavity 210 in the header 112 so as to be oriented transversely to a longitudinal axis of the bore 114, 116, 118. In some examples, the set screw 124 can be configured to be tightened and loosed using a wrench, such as an Allen-type wrench. As noted above, there can be fluid or contaminant flow into the cavity when tightening the set screw 124 for securing the terminal 17 in the header 112.

In this example, a gel 202 can be inserted into the cavity 210 around a head 126 of the set screw 124. A rubber grommet 204, such as a semi-closed rubber grommet, is positioned in the cavity 210 and positioned above the gel 202. The rubber grommet 204 is closer to an outer surface and farther from the set screw 124 than the gel 202. The rubber grommet 204 includes a slit 206 to allow a wrench through to reach the set screw 124. After the wrench is pulled back out, the gel 202 reflows and seals slit 206 area. Thus, the gel 202 is reflowable so as to backfill the cavity 210 after the wrench removed from the cavity 210. The wrench is first inserted through the slit in the rubber grommet 204 and then through the gel 202 and into the set screw 124.

The gel 202 is configured to fill the cavity 210 such that there is no airspace within the cavity 210 between the rubber grommet 204 and the set screw 124. In one example, the gel 202 can be a low viscosity gel. For example, the gel can have a viscosity of less than about 1,000 cP (1,000 mPa*s). In one embodiment, the gel can have a viscosity in the range of about 10 mPa*s to about 1,000 mPa*s. In one embodiment, the gel can have a viscosity in the range of about 100 mPa*s to about 1,000 mPa*s. For example, the gel 202 can be a medical grade silicone gel, such as NuSil Gel-8100 or MED1-6300 provided by NuSil Technology.

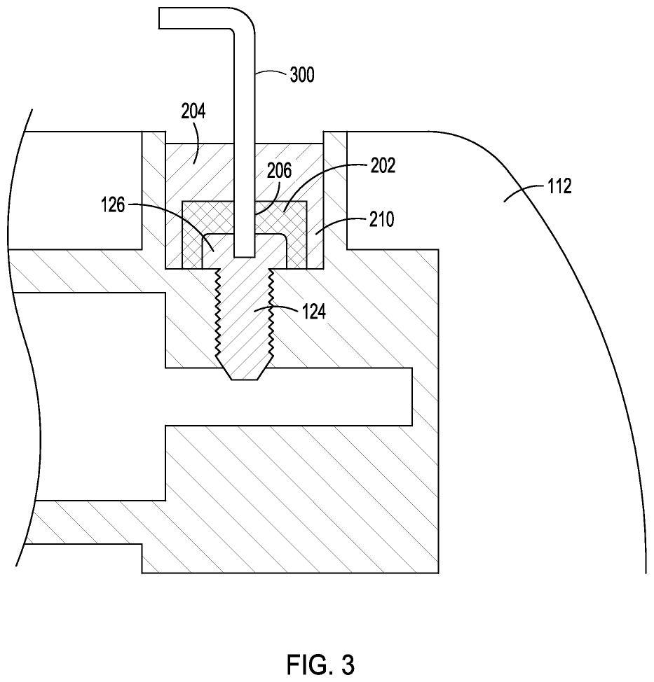

FIG. 3 shows the side view of FIG. 2 with a wrench 300 inserted into the cavity 210, and into the set screw 124, in accordance with one embodiment.

Here, when the wrench 300 is inserted into the cavity 210, the wrench 300 passes through the slit 206 and through the gel 202 into the set screw head 126 while the gel 202 flows around the wrench 300 so as to keep a tight seal on the cavity 201.

Then, when the wrench 300 is pulled out of the cavity 210, the situation reverts to the situation shown in FIG. 2, where the gel 202 backflows and fills the cavity 210 and seals the slit 206 and the cavity 210. Accordingly, the gel 202 prevents the influx of fluid or contaminants into the cavity 210 during and after the set screw fastening process.

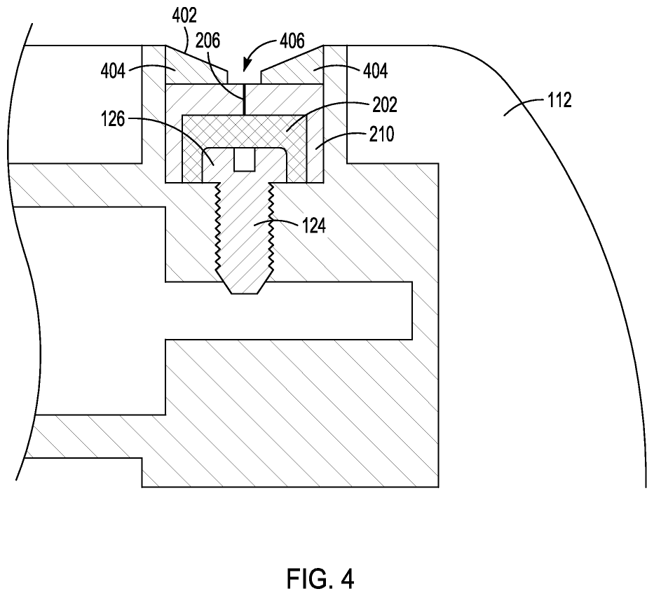

FIG. 4 shows a side view of a header bore in accordance with one embodiment. Here, a wrench guiding cap 402 can be positioned in the cavity 210 over the gel 202. In this example, the wrench guiding cap 402 is positioned over the rubber grommet 204 and over the gel 202. The wrench guiding cap 402 can be a circular, hard plastic or metallic member positioned within the cavity 210 and configured to prevent core outs of rubber grommet 204 and the gel 202 when the wrench is being removed from the cavity 210. Further, the wrench guiding cap 402 can include sloping side walls 404 leading down to a through hole 406. The through hole 406 is sized to permit the wrench 300 to pass through into the cavity 210. Moreover, the through hole 406 is positioned directly over the head 126 of the set screw 124 so that the wrench 300 is guided right into the head 126 of the set screw 124.

The sloping side walls 404 provide a funnel shaped upper surface of the wrench guiding cap 402 that act as a funnel to guide the wrench 300 into the through hole 406 and straight through the slit 206 to the screw head 126 of the set screw 124. Thus, as the user applies the wrench, if the wrench is off center, the sloping side walls 404 will guide the wrench to the proper position.

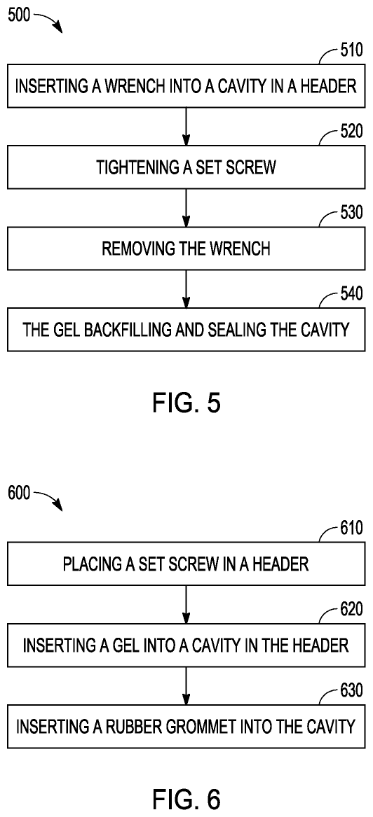

FIG. 5 depicts a method for fastening a set screw in a header. Referring also to FIGS. 2-4, the method (500) can include inserting a wrench into a cavity (510) in a header such that the wrench passes through a rubber grommet and then passes through a gel into a head of the set screw, tightening the set screw (520) using the wrench, removing the wrench (530) from the cavity, and the gel backfilling (540) and sealing the cavity.

As discussed above, the gel can be a low viscosity gel. The gel can have a viscosity of less than about 1,000 cP (1,000 mPa*s), and the gel can be a medical grade, silicone gel.

As discussed above, the method can further include providing a wrench guiding cap that acts as a funnel to guide the wrench. Moreover, the wrench guiding cap can help prevent core outs of the gel or the grommet when the wrench is removed from the cavity.

FIG. 6 shows a method (600) of manufacturing a header for an implantable device, in accordance with one embodiment. The method can include placing a set screw in a header (610), inserting a gel (620) into a cavity in the header over and around a head of the set screw, and inserting a rubber grommet into the cavity over the gel (630).

In various examples, inserting the gel can come before or after inserting the rubber grommet. For example, the gel can be inserted into the cavity and the grommet then inserted onto the cavity over the gel. Or the rubber grommet can be inserted into the cavity and then the gel can be injected through the slit in the rubber grommet to fill the area below the grommet and above the set screw.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device comprising:
a housing including electronic devices within the housing;
a header attached to the housing and including one or more bores configured to receive a lead; and
a set screw positioned in the header and configured to clamp to a lead inserted to the bore;
wherein, a gel is inserted into a cavity around a head of the set screw.

2. The implantable medical device of claim 1, including a semi-closed rubber grommet positioned in the cavity and positioned above the gel.

3. The implantable medical device of claim 1, wherein the gel is configured to fill the cavity.

4. The implantable medical device of claim 1, wherein the gel is a low viscosity gel.

5. The implantable medical device of claim 4, wherein the gel has a viscosity of less than about 1,000 cP (1,000 mPa*s).

6. The implantable medical device of claim 5, wherein the gel is a medical grade, silicone gel.

7. The implantable medical device of claim 1, wherein the set screw is configured to be tightened and loosed using a wrench.

8. The implantable medical device of claim 7, wherein the gel is reflowable so as to backfill the cavity after the wrench removed from the cavity.

9. The implantable medical device of claim 1, wherein the set screw is positioned in the cavity in the header so as to be oriented transversely to a longitudinal axis of the bore.

10. The implantable medical device of claim 1, further including a wrench guiding cap positioned in the cavity over the gel.

11. The implantable medical device of claim 10, wherein the wrench guiding cap includes a funnel shaped upper surface leading to a through hole in the wrench guiding cap.

12. A method for fastening a set screw in a header, the method comprising:
inserting a wrench into a cavity in a header such that the wrench passes through a rubber grommet and then passes through a gel into a head of the set screw;
tightening the set screw using the wrench;
removing the wrench from the cavity; and
the gel backfilling and sealing the cavity.

13. The method of claim 12, wherein the gel is a low viscosity gel.

14. The method of claim 13, wherein the gel has a viscosity of less than about 1,000 cP (1,000 mPa*s).

15. The method of claim 14, wherein the gel is a medical grade, silicone gel.

16. The method of claim 12, further including a wrench guiding cap positioned in the cavity over the gel.

17. The method of claim 16, wherein the wrench guiding cap includes a funnel shaped upper surface leading to a through hole in the wrench guiding cap.

18. A method of manufacturing a header for an implantable device, the method comprising:
placing a set screw in a header;
inserting a gel into a cavity over and around a head of the set screw, and
inserting a rubber grommet into the cavity over the gel.

19. The method of claim 18, wherein inserting the gel comes after inserting the rubber grommet.

20. The method of claim 18, wherein the gel is a low viscosity medical grade silicone gel.
